(19) United States
(12) Patent Application Publication
(10) Pub. No.: US 2018/0344645 A1
(43) Pub. Date: Dec. 6, 2018

(54) PREPARTION OF NANOCRYSTALS AND NANAOPARTICLES OF NARROW DISTRIBUTION AND USES THEREOF

(71) Applicant: Purdue Research Foundation, West Lafayette, IN (US)

(72) Inventors: Tonglei Li, West Lafayette, IN (US); Wei Gao, Dandong (CN)

(73) Assignee: Purdue Research Foundation, West Lafayette, IN (US)

(21) Appl. No.: 16/000,986

(22) Filed: Jun. 6, 2018

Related U.S. Application Data
(60) Provisional application No. 62/515,571, filed on Jun. 6, 2017.

(51) Int. Cl. A61K 9/14; A61K 36/899; A61K 36/81; A61K 9/51; A61K 9/19

(52) U.S. Cl. CPC A61K 9/145; A61K 36/899; A61K 36/81; A61K 9/5169; A61K 9/19; A61K 9/5161; A61K 9/5123

(57) ABSTRACT

The present invention generally relates to preparation methods of nanoparticles or nanocrystals of narrowly distributed particle sizes of a poorly water soluble material. In particular, this invention is an effective integration of the bottom-up and top-down preparation methods of nanoparticles and nanocrystals of a poorly water soluble material or active pharmaceutical ingredient into a single process, which affords an efficient and simple process with a product of improved delivery and therapeutic effects.

### CROSS REFERENCE TO RELATED APPLICATIONS

This present patent application relates to and claims the priority benefit of U.S. Provisional Application Ser. No. 62/515,571, filed Jun. 6, 2017, the content of which is hereby incorporated by reference in its entirety.

### TECHNICAL FIELD

The present disclosure generally relates to preparation methods of nanoparticles or nanocrystals of narrowly distributed particle sizes. In particular, this disclosure relates to a process for the preparation of nanoparticles or nanocrystals of a material or pharmaceutical ingredient (API) with narrowly distributed particle sizes for improved delivery and therapeutic effects.

### BACKGROUND

This section introduces aspects that may help facilitate a better understanding of the disclosure. Accordingly, these statements are to be read in this light and are not to be understood as admissions about what is or is not prior art.

Nanotechnology has sparked a rapidly growing interest as it promises to solve a number of issues associated with conventional therapeutic agents, such as poor water solubility for some anticancer drugs, lack of targeting capability, nonspecific distribution, systemic toxicity, and low therapeutic index (T. Sun, et al., “Engineered Nanoparticles for Drug Delivery in Cancer Therapy,” Angew. Chem. Int'l Ed. 2014, 53, Pages 12320-12364).

Delivering a drug substance as nanocrystals (NCs) becomes an enabling formulation technology for drug development. It requires no carrier design and thus circumvents limitations typically caused with utilization of solubilizing/encapsulating chemicals, such as instability and inherent toxic side effects. Importantly, a nanocrystal formulation offers 100% drug loading when no surfactant is used. When the surface of nanocrystals is coated, the usage of surfactants is still minimal. The physical stability offered by drug nanocrystals permits reliable pharmacokinetic characters and predictable in vivo performance.

Nanosized materials (having a size in the range of from 1 nm to 1,000 nm, referred to herein as the nanorange) have particular physical-chemical properties. These properties can be exploited for the improved delivery of poorly soluble actives in pharma (drugs, diagnostics), cosmetics (cosmetic actives) and nutrition (e.g. nutraceuticals) technology. Amongst these properties are increased saturation solubility, increased dissolution rate, adhesiveness to surfaces/membranes in the body, and the small size itself allowing for example to use certain administration routes, e.g. intravenous injection. The particles can be crystalline (so called nanocrystals), or can be amorphous (so called amorphous nanoparticles), or can be a mixture of crystalline and amorphous (partially crystalline) particles. An overview about these nanosized materials is given in the reviews by C. M. Keck and R. H. Müller (Müller, R. H., C. M. Keck, J Biotechnol. 2004. 113(1-3): p. 151-70.; Müller, R H, Gohla, S, Keck, C M, Eur J Pharm Biopharm 2011; 78: 1-9). The nanosized materials are typically produced in a liquid by wet milling. A suspension of nanosized material in a liquid is called nanosuspension.

There are basically two approaches to create these nanosized materials, the “bottom-up” technologies and the “top-down” technologies (Keck, U.S. Pat. No. 9,040,105, 2015). A top-down approach utilizes mechanical means to comminute regular crystal samples. Typical methods include wet ball milling (WBM) and high pressure homogenization (HPH). WBM (also referred to as pearl or bead milling) is by far the most frequently used production method for drug nanocrystals in the pharmaceutical industry. Coarse drug substance, or prepared micronized suspension, is added to a mill containing milling pearls or beads moved by an agitator. The drug crystals are broken through abrasion and cleavage. HPH is likely the second mostly important approach for the drug nanocrystals production.

Microfluidization and piston gap homogenization are two realizations of HPH. In microfluidization, drug crystals are fragmented in a high pressured air jet induced by collision of two fluid streams. In piston-gap homogenization, a drug crystal suspension is forced through a mechanical gap, sheared by cavitation forces and particle collision under the high pressure condition. Drug nanocrystals generated by these methods, however, contain chemical and physical impurities—the former is due to breakage of milling beads and the latter due to the amorphous content created by the mechanical processes. These nanocrystals may not be suitable for parental delivery routes; the commercial products of drug nanocrystals prepared by top-down approaches are mainly for oral delivery.

On the other hand, the bottom-up approach generates drug nanocrystals through crystallization from a liquid environment containing dissolved drug molecules by a process of precipitation (Sucker, et al., GB Patent 2200048, 1988; GB Patent 2269536, 1994). The antisolvent method is perhaps the mostly used bottom-up process. It starts with the drug dissolved in a (organic) solvent, followed by introduction into a poor solvent (most likely, water). Reduction in solubility in the new solvent environment triggers nucleation leading to development drug crystals. By controlling the crystallization conditions, it is possible to control and maintain the particle size of growing nuclei. Yet, a different drug substance may require a completely unique set of crystallization conditions in order to produce nanocrystals. Scaling up of the anti-solvent method remains a tremendous challenge as it is difficult to scale up the nucleation conditions, such as sonication intensity and distribution.

### BRIEF SUMMARY OF INVENTION

The present disclosure generally relates to preparation methods of nanoparticles or nanocrystals of narrowly distributed particle sizes. In particular, this disclosure relates to a novel scale up process for the preparation of nanoparticles or nanocrystals of a material or an active pharmaceutical ingredient (API) for improved delivery and therapeutic effects.

In some aspects, this invention is an effective integration of bottom-up and top-down preparation methods of nanoparticles and nanocrystals into a single process, which affords an efficient and simple process with a product of narrowly distributed particle sizes.

In some aspects, this invention is an effective integration of bottom-up and top-down preparation methods of nanoparticles or nanocrystals of a material or an API into a single process, which affords narrowly distributed particle sizes and improved therapeutic effects.

In other aspects, described herein is a process for the preparation of nanoparticles or nanocrystals of a material or an API, the process comprises the steps of
a. dissolving said material in an organic solvent to afford an organic solution;
b. adding the organic solution of said material to a chilled aqueous system in a container with a plurality of inlets/outlets under sonication and vigorous stirring using a rotor-stator mixer to give a suspension;
c. passing said suspension through a said outlet to a piston-gap high pressure homogenizer to produce a first cycle of nanocrystals or amorphous nanoparticles of said material;
d. returning the first cycle of nanocrystals or amorphous nanoparticles of said material through a said inlet back to the chilled aqueous system in the container under sonication and vigorous stirring; and
e. continuing steps c. and d. until nanocrystals or amorphous nanoparticles of said material reach desired sizes and distribution.

In some aspects, described herein is a process for the preparation of nanoparticles or nanocrystals of a material or an API, the process further comprises a step adding a surfactant comprising polysorbate 80, sorbitan, sodium dodecyl sulfate, a quarternary ammonium salt, or a pyridinium cationic salt.

In some other aspects, chilling in step b. above is provided by an ice-water bath equipped with an ultrasonicating system.

In some other aspects, described herein is a process for the preparation of nanoparticles or nanocrystals of a material or an API, wherein said API comprises albendazole, danazol, ketoconazole, itrconazole, atovaquone, troglitazone, valsartan, nimesulide, loratadine, griseofulvin, felodipine, paclitaxel, probucol, ubiquinone, cefixime frusemide, salicylic acid, ketoprofen, tinidazole, aceclofenac, hydrochlorothiazide, ciprofloxacin, ibuprofen, or nevirapine.

In some aspects, described herein is a pharmaceutical composition comprising nanocrystals or nanoparticles of an API prepared according to the process disclosed herein, together with one or more pharmaceutically acceptable carriers, diluents, and excipients.

In some other aspects, described herein is a pharmaceutical composition comprising nanocrystals or nanoparticles of an API with a poor aqueous solubility prepared according to the process disclosed herein, together with one or more pharmaceutically acceptable carriers, diluents, and excipients.

In some aspects, described herein is a pharmaceutical composition comprising nanocrystals or nanoparticles of albendazole, danazol, ketoconazole, itrconazole, atovaquone, troglitazone, valsartan, nimesulide, loratadine, griseofulvin, felodipine, paclitaxel, probucol, ubiquinone, cefixime frusemide, salicylic acid, ketoprofen, tinidazole, aceclofenac, hydrochlorothiazide, ciprofloxacin, ibuprofen, or nevirapine, prepared according to the process disclosed herein, together with one or more pharmaceutically acceptable carriers, diluents, and excipients.

In some aspects, described herein is a pharmaceutical composition comprising nanocrystals or nanoparticles of paclitaxel or itraconazole prepared according to the process disclosed herein, together with one or more pharmaceutically acceptable carriers, diluents, and excipients.

Yet in some other aspects, described herein is a pharmaceutical composition comprising nanocrystals or nanoparticles of an API manufactured according to the process of:
a. dissolving said material or API in an organic solvent to afford an organic solution;
b. adding the organic solution of said API to a chilled aqueous system in a container with a plurality of inlets/outlets under sonication and vigorous stirring using a rotor-stator mixer to give a suspension;
c. passing said suspension through a said outlet to a piston-gap high pressure homogenizer to produce a first cycle of nanocrystals or amorphous nanoparticles of said API;
d. returning the first cycle of nanocrystals or amorphous nanoparticles of said API through a said inlet back to the chilled aqueous system in the container under sonication and vigorous stirring; and
e. continuing steps c. and d. until nanocrystals or amorphous nanoparticles of said API reach desired sizes and distribution.

### BRIEF DESCRIPTION OF THE FIGURES

The above and other objects, features, and advantages of the present invention will become more apparent when taken in conjunction with the following description and drawings.

FIG. 1 shows schematic diagram of the scaling up method disclosed herein.

FIG. 2A shows scanning electron microscope (SEM) images of paclitaxel nanocrystals prepared according to the method disclosed herein at a scale bar of 1 μm.

FIG. 2B shows scanning electron microscope (SEM) images of paclitaxel nanocrystals prepared according to the method disclosed herein at a scale bar of 500 nm.

FIG. 3A-3C show scanning electron microscope (SEM) images of itraconazole nanocrystals prepared according to the method disclosed herein at a scale of 5 μm (FIG. 3A), 2 μm (FIG. 3B), and 1 μm (FIG. 3C), respectively.

### DETAILED DESCRIPTION

For the purposes of promoting an understanding of the principles of the present disclosure, references will now be made to the embodiments illustrated in the drawings, and specific language will be used to describe the same. It will nevertheless be understood that no limitation of the scope of this disclosure is thereby intended.

The present disclosure generally relates to preparation methods of nanoparticles or nanocrystals of narrowly distributed particle sizes. In particular, this disclosure relates to a novel scale up process for the preparation of nanoparticles or nanocrystals of a material or active pharmaceutical ingredient (API) for improved delivery and therapeutic effects.

In some illustrative embodiments, this invention is an integration of bottom-up and top-down preparation methods of nanoparticles and nanocrystals into a single process, which affords an efficient and simple process with a product of narrowly distributed particle sizes.

In some illustrative embodiments, this invention is an effective integration of bottom-up and top-down preparation methods of nanoparticles or nanocrystals of an API into a single process, which affords narrowly distributed particle sizes and improved therapeutic effects.

In some other illustrative embodiments, described herein is a process for preparing nanocrystals or nanoparticles of a material comprising the steps of
a. dissolving said material in an organic solvent to afford an organic solution;
b. adding the organic solution of said material to a chilled aqueous system in a container with a plurality of inlets/outlets under sonication and vigorous stirring using a rotor-stator mixer to produce a suspension;
c. passing said suspension through a said outlet to a piston-gap high pressure homogenizer to produce a first cycle of nanocrystals or amorphous nanoparticles of said material;
d. returning the first cycle of nanocrystals or amorphous nanoparticles of said material through a said inlet back to the chilled aqueous system in the container under sonication and vigorous stirring; and
e. continuing steps c. and d. until nanocrystals or amorphous nanoparticles of said material reach desired sizes and distribution.

In some other illustrative embodiments, described herein is a process for preparing nanocrystals or nanoparticles of a material disclosed herein, wherein said chilling in step b. is provided by an ice-water bath equipped with an ultrasonicating system.

In some other illustrative embodiments, described herein is a process for preparing nanocrystals or nanoparticles of a material disclosed herein, wherein said chilled aqueous system is chilled by placing in an ultrasonic bath filled with ice-water.

In some other illustrative embodiments, described herein is a process for preparing nanocrystals or nanoparticles of a material disclosed herein, wherein said rotational speed of stirring in step b provide by a rotor-stator ranges from about 200 rpm to about 20,000 rpm.

In some other illustrative embodiments, described herein is a process for preparing nanocrystals or nanoparticles of a material disclosed herein, wherein said organic solution is made of said material and an organic solvent, wherein said material is highly soluble in the organic solvent.

In some other illustrative embodiments, described herein is a process for preparing nanocrystals or nanoparticles of a material disclosed herein, wherein said organic solvent is water miscible.

In some other illustrative embodiments, described herein is a process for preparing nanocrystals or nanoparticles of a material disclosed herein, wherein said organic solvent is ethanol, acetone or acetonitrile.

In some other illustrative embodiments, described herein is a process for preparing nanocrystals or nanoparticles of a material disclosed herein, wherein said material is a compound poorly soluble in water or an aqueous medium.

In some other illustrative embodiments, described herein is a process for preparing nanocrystals or nanoparticles of a material disclosed herein, wherein said poorly water soluble compound is an active pharmaceutical ingredient (API) comprising albendazole, danazol, ketoconazole, itrconazole, atovaquone, troglitazone, valsartan, nimesulide, loratadine, griseofulvin, felodipine, paclitaxel, probucol, ubiquinone, cefixime frusemide, salicylic acid, ketoprofen, tinidazole, aceclofenac, hydrochlorothiazide, ciprofloxacin, ibuprofen, or nevirapine.

In some other illustrative embodiments, described herein is a process for preparing nanocrystals or nanoparticles of a material disclosed herein, wherein said process further comprising a step adding a surfactant, wherein said surfactant comprises polysorbate 80, sorbitan, sodium dodecyl sulfate, a quaternary ammonium, or a pyridinium cationic surfactant.

In some other illustrative embodiments, described herein is a process for preparing nanocrystals or nanoparticles of a material disclosed herein, wherein said process further comprising a step adding a PEG-PPG-PEG (polyethylene glycol-polypropylene glycol-polyethylene glycol) copolymer.

In some other illustrative embodiments, described herein is a nanocrystal or nanoparticle prepared according to the process disclosed herein.

In some other illustrative embodiments, described herein is a pharmaceutical composition comprising nanocrystals or nanoparticles of an active pharmaceutical ingredient prepared according to the process disclosed herein.

In some other illustrative embodiments, described herein is a pharmaceutical composition comprising nanocrystals or nanoparticles of albendazole, danazol, ketoconazole, itrconazole, atovaquone, troglitazone, valsartan, nimesulide, loratadine, griseofulvin, felodipine, paclitaxel, probucol, ubiquinone, cefixime frusemide, salicylic acid, ketoprofen, tinidazole, aceclofenac, hydrochlorothiazide, ciprofloxacin, ibuprofen, or nevirapine, prepared according to the process disclosed herein.

In some illustrative embodiments, described herein is a pharmaceutical composition comprising nanocrystals or nanoparticles of paclitaxel prepared according to the process disclosed herein, together with one or more pharmaceutically acceptable carriers, diluents, and excipients.

In some illustrative embodiments, described herein is a pharmaceutical composition comprising nanocrystals or nanoparticles of itraconazole prepared according to the process disclosed herein, together with one or more pharmaceutically acceptable carriers, diluents, and excipients.

In some other illustrative embodiments, described herein is a method for treating a patient with a disease comprising the step of administering a therapeutically effective amount of a pharmaceutical composition disclosed herein to the patient in need of relief from said disease.

In some illustrative embodiments, described herein is a method for treating a patient with a disease comprising the step of administering a therapeutically effective amount of a pharmaceutical composition disclosed herein, together with a therapeutically effective amount of one or more other compounds, to the patient in need of relief from said disease.

In some illustrative embodiments, described herein is a pharmaceutical composition comprising nanocrystals or nanoparticles of an active pharmaceutical ingredient (API) manufactured according to the steps of
a. dissolving said material or API in an organic solvent to afford an organic solution;
b. adding the organic solution of said API to a chilled aqueous system in a container with a plurality of inlets/outlets under sonication and vigorous stirring using a rotor-stator mixer to give a suspension;
c. passing said suspension through a said outlet to a piston-gap high pressure homogenizer to produce a first cycle of nanocrystals or amorphous nanoparticles of said API;
d. returning the first cycle of nanocrystals or amorphous nanoparticles of said API through a said inlet back to the chilled aqueous system in the container under sonication and vigorous stirring; and
e. continuing steps c. and d. until nanocrystals or amorphous nanoparticles of said API reach desired sizes and distribution.

In some illustrative embodiments, described herein is a pharmaceutical composition manufactured according to the process disclosed herein, wherein said manufacturing process further comprising a step adding a surfactant, wherein said surfactant comprises polysorbate 80, sorbitan, sodium dodecyl sulfate, a quaternary ammonium, or a pyridinium cationic surfactant.

In some illustrative embodiments, described herein is a pharmaceutical composition manufactured according to the process disclosed herein, wherein said API is a poorly water-soluble compound comprising albendazole, danazol, ketoconazole, itrconazole, atovaquone, troglitazone, valsartan, nimesulide, loratadine, griseofulvin, felodipine, paclitaxel, probucol, ubiquinone, cefixime frusemide, salicylic acid, ketoprofen, tinidazole, aceclofenac, hydrochlorothiazide, ciprofloxacin, ibuprofen, or nevirapine.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, or within 99% of a stated value or of a stated limit of a range.

The term “patient” includes human and non-human animals such as companion animals (dogs and cats and the like) and livestock animals. Livestock animals are animals raised for food production. The patient to be treated is preferably a mammal, in particular a human being.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

It is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender, and diet of the patient: the time of administration, and rate of excretion of the specific compound employed, the duration of the treatment, the drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosage may be single or divided, and may be administered according to a wide variety of dosing protocols, including q.d., b.i.d., t.i.d., or even every other day, once a week, once a month, and the like. In each case the therapeutically effective amount described herein corresponds to the instance of administration, or alternatively to the total daily, weekly, or monthly dose.

As used herein, the term “therapeutically effective amount” refers to that amount of active compound or pharmaceutical agent that elicits the biological or medicinal response in a tissue system, animal or human that is being sought by a researcher, veterinarian, medical doctor or other clinicians, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment.

As used herein, the term “therapeutically effective amount” refers to the amount to be administered to a patient, and may be based on body surface area, patient weight, and/or patient condition. In addition, it is appreciated that there is an interrelationship of dosages determined for humans and those dosages determined for animals, including test animals (illustratively based on milligrams per meter squared of body surface) as described by Freireich, E. J., et al., Cancer Chemother. Rep. 1966, 50 (4), 219, the disclosure of which is incorporated herein by reference. Body surface area may be approximately determined from patient height and weight (see, e.g., Scientific Tables, Geigy Pharmaceuticals, Ardley, New York, pages 537-538 (1970)). A therapeutically effective amount of the compounds described herein may be defined as any amount useful for inhibiting the growth of (or killing) a population of malignant cells or cancer cells, such as may be found in a patient in need of relief from such cancer or malignancy. Typically, such effective amounts range from about 5 mg/kg to about 500 mg/kg, from about 5 mg/kg to about 250 mg/kg, and/or from about 5 mg/kg to about 150 mg/kg of compound per patient body weight. It is appreciated that effective doses may also vary depending on the route of administration, optional excipient usage, and the possibility of co-usage of the compound with other conventional and non-conventional therapeutic treatments, including other anti-tumor agents, radiation therapy, and the like.

Poorly water soluble drugs. In generally, “poorly water soluble” is defined as 10,000 part solvent to dissolve 1 part of solute (Ketan T. Savjani, et al., “Drug Solubility: Importance and Enhancement Techniques.” ISRN Pharm. 2012; 2012: 195727). In other words, a poorly soluble drug has solubility less than 0.1 mg/mL. These drugs may include: albendazole, danazole, ketoconazole, itrconazole, atovaquone, troglitazone, valsartan, nimesulide, loratadine, griseofulvin, felodipine, probucol, ubiquinone, cefixime frusemide, salicylic acid, ketoprofen, tinidazole, aceclofenac, hydrocholthiazide,ofloxacin, ibufrofen, and nevirapine, A recent report provided a more comprehensive list of those poorly soluble drugs (Sandeep Kalepu, et al., Acta Pharmaceutica Sinica B, 2015, Vol. 5(5), 442-453). The strategies disclosed herein may find applications to all of those poorly soluble drug compounds for improved delivery and better therapeutic outcomes.

The present invention may be better understood in light of the following non-limiting method examples with reference to the accompanying drawings.

### EXAMPLE 1

### Preparation of Paclitaxel Nanocrystals (PTX NCs)

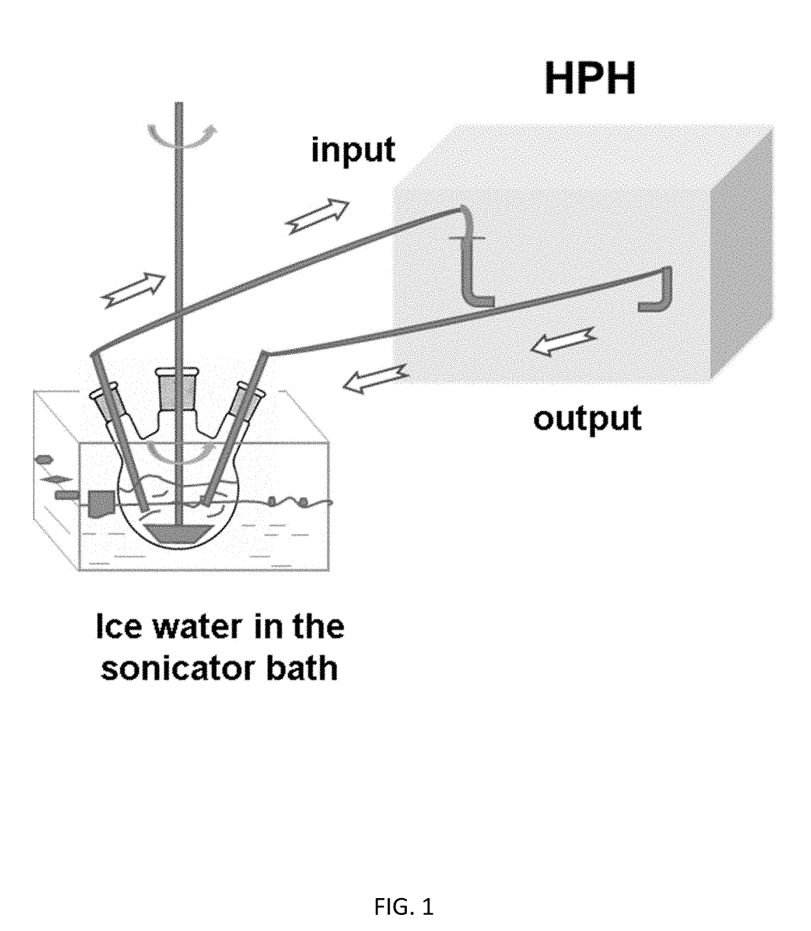

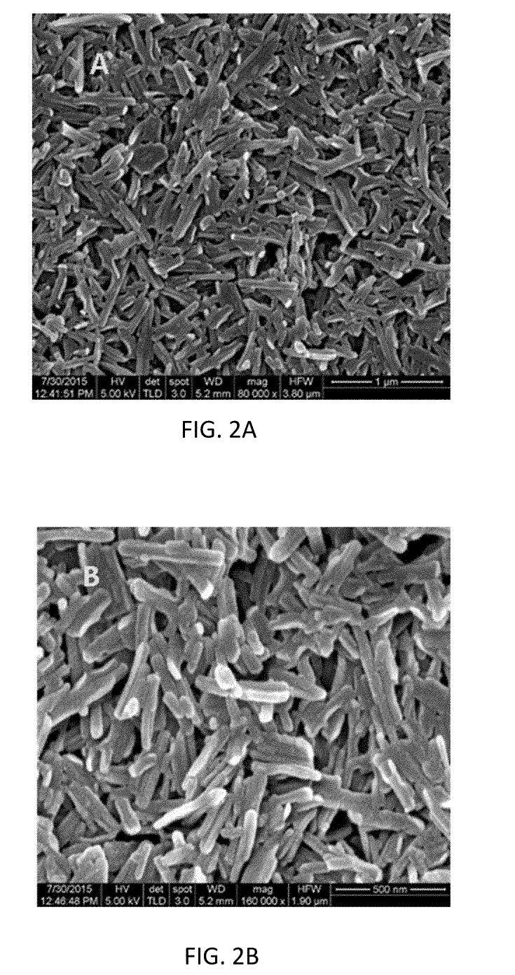

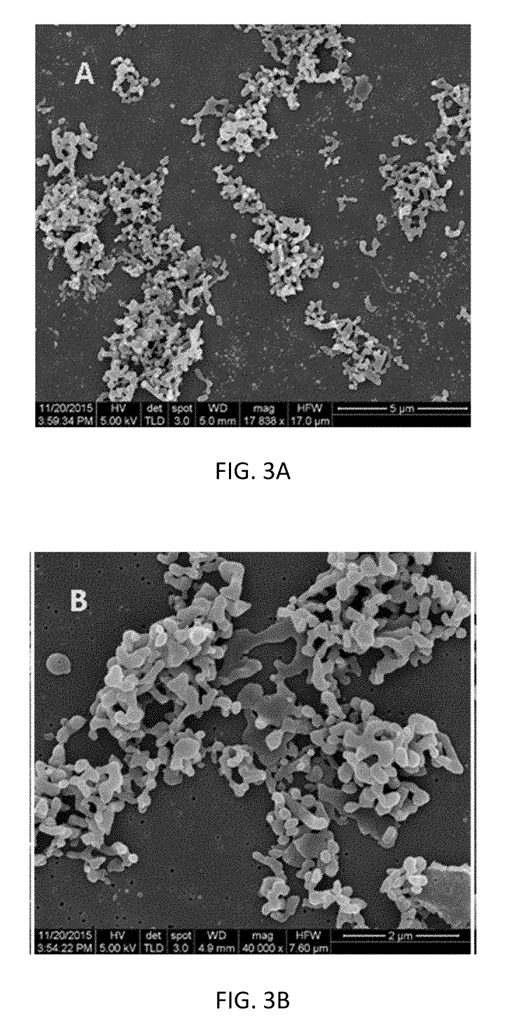

AS shown in FIG. 1, the process described herein is a one-step procedure for the preparation of PTX NCs with our invented method. To a 40 mL of paclitaxel (3 mg/mL) ethanol solution was introduced to 800 mL double distilled water in a 3-neck flask that was placed in a sonication ice-water bath. The solution was then agitated with a stirrer shaft at 1200 rpm, through a high-pressure homogenizer (HPH) at a pressure of 1400 bar for 5 min. Particle size and size distribution of the nanocrystals were measured by dynamic light scattering (DLS) using a Malvern Zetasizer Nano ZS instrument at 25° C. The morphology of PTX NCs were examined using a scanning electron microscope (SEM). The average particle size of PTX NCs was 268±11 nm, PDI was 0.11±0.1. As shown in FIG. 2, the nanocrystals exhibited well-defined, rod-like morphologies.

### EXAMPLE 2

### Preparation of Itraconazole Nanocrystals (ITC NCs)

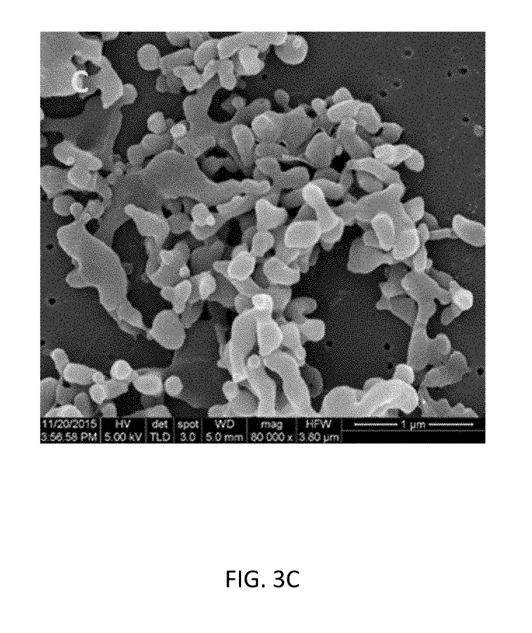

To a 20 mL of itraconazole (1 mg/mL) acetone solution was introduced to 600 mL double distilled water in the 3-neck flask with a sonication in ice-water bath. The mixture solution was agitated with a stirrer shaft at 1200 rpm, through HPH at a pressure of 1500 bar for 8 min. Particle size and size distribution of the nanocrystals were measured by DLS at 25° C. The morphology of ITC NCs were examined using a scanning electron microscope. The average particle size of ITC NCs was 529±16 nm, PDI was 0.19±0.1. As shown in FIG. 3A-FIG. 3C, the nanocrystals of itraconazole exhibited square-ish and roundish morphologies.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

What is claimed is:

1. A process for preparing nanocrystals or nanoparticles of a material comprising the steps of
a. dissolving said material in an organic solvent to afford an organic solution;
b. adding the organic solution of said material to a chilled aqueous system in a container with a plurality of inlets/outlets under sonication and vigorous stirring using a rotor-stator mixer to produce a suspension;
c. passing said suspension through a said outlet to a piston-gap high pressure homogenizer to produce a first cycle of nanocrystals or amorphous nanoparticles of said material;
d. returning the first cycle of nanocrystals or amorphous nanoparticles of said material through a said inlet back to the chilled aqueous system in the container under sonication and vigorous stirring; and
e. continuing steps c. and d. until nanocrystals or amorphous nanoparticles of said material reach desired sizes and distribution.

2. The process of claim 1, wherein chilling in step b. is provided by an ice-water bath equipped with an ultrasonicating system.

3. The process of claim 1, wherein the chilled aqueous system is chilled by placing in an ultrasonic bath filled with ice-water.

4. The process of claim 1, wherein rotational speed of stirring in step b provide by a rotor-stator ranges from about 200 rpm to about 20,000 rpm.

5. The process of claim 1, wherein said organic solution is made of said material and an organic solvent, wherein said material is highly soluble in the organic solvent.

6. The process of claim 1, wherein said organic solvent is water miscible.

7. The process of claim 6, wherein said organic solvent is ethanol, acetone or acetonitrile.

8. The process of claim 1, wherein said material is a compound poorly soluble in water or an aqueous medium.

9. The process of claim 8, wherein said compound is an active pharmaceutical ingredient (API) comprising albendazole, danazol, ketoconazole, itrconazole, atovaquone, troglitazone, valsartan, nimesulide, loratadine, griseofulvin, felodipine, paclitaxel, probucol, ubiquinone, cefixime frusemide, salicylic acid, ketoprofen, tinidazole, aceclofenac, hydrochlorothiazide, ciprofloxacin, ibuprofen, or nevirapine.

10. The process according to claim 1, further comprising a step adding a surfactant, wherein said surfactant comprises polysorbate 80, sorbitan, sodium dodecyl sulfate, a quaternary ammonium, or a pyridinium cationic surfactant.

11. The process according to claim 1, further comprising a step adding a PEG-PPG-PEG (polyethylene glycol-polypropylene glycol-polyethylene glycol) copolymer.

12. A nanocrystal or nanoparticle prepared according to the process of claim 1.

13. A pharmaceutical composition comprising nanocrystals or nanoparticles of an active pharmaceutical ingredient prepared according to the process of claim 1.

14. A pharmaceutical composition comprising the nanocrystals or nanoparticles of paclitaxel prepared according to the process of claim 1.

15. A pharmaceutical composition comprising the nanocrystals or nanoparticles of itraconazole prepared according to the process of claim 1.

16. A pharmaceutical composition comprising the nanocrystals or nanoparticles prepared according to the process of claim 1, together with one or more pharmaceutically acceptable carriers, diluents, and excipients.

17. A method for treating a patient, the method comprising the step of administering a therapeutically effective amount of a pharmaceutical composition of claim 16 to the patient in need of relief.

18. A pharmaceutical composition comprising nanocrystals or nanoparticles of an active pharmaceutical ingredient (API) manufactured according to the steps of
a. dissolving said material or API in an organic solvent to afford an organic solution;
b. adding the organic solution of said API to a chilled aqueous system in a container with a plurality of inlets/outlets under sonication and vigorous stirring using a rotor-stator mixer to give a suspension;
c. passing said suspension through a said outlet to a piston-gap high pressure homogenizer to produce a first cycle of nanocrystals or amorphous nanoparticles of said API;
d. returning the first cycle of nanocrystals or amorphous nanoparticles of said API through a said inlet back to the chilled aqueous system in the container under sonication and vigorous stirring; and
e. continuing steps c. and d. until nanocrystals or amorphous nanoparticles of said API reach desired sizes and distribution.

19. The pharmaceutical composition according to claim 18, wherein said manufacturing process further comprising a step adding a surfactant, wherein said surfactant comprises polysorbate 80, sorbitan, sodium dodecyl sulfate, a quaternary ammonium, or a pyridinium cationic surfactant.

20. The pharmaceutical composition according to claim 18, wherein said API is a poorly water-soluble compound comprising albendazole, danazol, ketoconazole, itrconazole, atovaquone, troglitazone, valsartan, nimesulide, loratadine, griseofulvin, felodipine, paclitaxel, probucol, ubiquinone, cefixime frusemide, salicylic acid, ketoprofen, tinidazole, aceclofenac, hydrochlorothiazide, ciprofloxacin, ibuprofen, or nevirapine.
